(19) United States
(12) Patent Application Publication
(10) Pub. No.: US 2022/0362557 A1
(43) Pub. Date: Nov. 17, 2022

(54) DETECTION OF INFECTION BASED ON TEMPERATURE AND IMPEDANCE

(71) Applicant: Medtronic, Inc., Minneapolis, MN (US)

(72) Inventors: Shailesh Kumar V. Musley, Brooklyn Park, MN (US); Maneesh Shrivastav, Blaine, MN (US); Abhijit Kadrolkar, St. Paul, MN (US); Todd J. Sheldon, North Oaks, MN (US); Vinod Sharma, Maple Grove, MN (US)

(21) Appl. No.: 17/663,350

(22) Filed: May 13, 2022

Related U.S. Application Data
(60) Provisional application No. 63/188,288, filed on May 13, 2021.

(51) Int. Cl. A61N 1/365; A61N 1/372; A61B 5/00; A61B 5/053; A61B 5/07

(52) U.S. Cl. CPC A61N 1/36521; A61N 1/37258; A61B 5/0031; A61B 5/053; A61B 5/076; A61B 5/686

(57) ABSTRACT

A system comprises an implantable medical device configured to generate temperature data and impedance data associated with temperature and impedance of a patient proximate to the implantable medical device. The system further comprises processing circuitry configured to determine whether a first one or more infection criteria are satisfied by temperature data and impedance data generated by the implantable medical device during a first time interval, wherein the first one or more infection criteria include at least one criterion indicative of decreased impedance, determine whether a second one or more infection criteria are satisfied by the temperature data and impedance data generated by the implantable medical device during a second time interval subsequent to the first time interval, wherein the second one or more infection criteria include at least one criterion indicative of increased impedance, and output, based on satisfaction of the first and second infection criteria, an indication of infection.

This application claims the benefit of U.S. Provisional Application Ser. No. 63/188,288, filed May 13, 2021, the entire content of which is incorporated herein by reference.

### FIELD

The disclosure relates to implantable medical devices and, more particularly, to techniques for monitoring infection, such as an infection in proximity to medical devices implanted in patients, a systemic infection, etc.

### BACKGROUND

Infection associated with implantation of medical devices is a serious health and economic concern. Infections associated with implanted medical devices are not very common due to care and precautions taken during surgical implantation of the devices. However, when an infection associated with an implanted medical device does occur, explanting the device is often the only appropriate course of action.

Some implantable medical devices (IMDs), such as cardiac pacemakers, cardioverter/defibrillators having pacing capabilities, cardiac or other physiological monitors, neurostimulators other electrical stimulators, cochlear implants, etc., include a housing that is implanted at a surgically prepared site, referred to as a “pocket.” In some cases, associated devices, such as elongated medical electrical leads or drug delivery catheters, extend from the pocket to other subcutaneous sites or deeper into the body to organs or other implantation sites.

Surgical preparation and implantation are conducted in a sterile field, and the IMD components are packaged in sterile containers sterilized prior to introduction into the sterile field or implanted in anti-bacterial envelopes. However, despite these precautions, there always is a risk of introduction of microbes into the pocket. Surgeons therefore typically apply disinfectant or antiseptic agents to the skin at the surgical site prior to surgery, directly to the site before the incision is closed, and prescribe oral antibiotics for the patient to ingest during recovery.

Despite these precautions, infections can occur. In addition, once the pocket becomes infected, the infection can migrate along the lead or catheter to locations in which the lead or catheter is implanted and result in systemic infection. Removal of a chronically implanted lead or catheter, e.g., in response to such an infection, can be complicated. Accordingly, aggressive systemic drug treatment is prescribed to treat such infections.

### SUMMARY

This disclosure describes techniques for detecting infections early, e.g., device pocket infections, based on measurements of temperature and impedance of a patient proximate to an implantable medical device (IMD). The IMD may be configured to generate temperature data and impedance data based on the measurements. Processing circuitry, e.g., of the IMD and/or a computing devices configured to communicate with the IMD, may determine whether infection criteria are satisfied by the temperature data and the impedance data, and output an indication of infection based on satisfaction of the criteria. The infection criteria may include at least one criterion indicative of decreased impedance during a first time interval and at least one criterion indicative of increased impedance during a second time interval subsequent to the first time interval. The techniques described herein may allow early detection of infections, which may allow for earlier intervention, resulting in fewer device explants.

In one example, a system comprises an implantable medical device configured to generate temperature data and impedance data associated with temperature and impedance of a patient proximate to the implantable medical device. The system further comprises processing circuitry configured to: determine whether a first one or more infection criteria are satisfied by temperature data and impedance data generated by the implantable medical device during a first time interval, wherein the first one or more infection criteria include at least one criterion indicative of decreased impedance; determine whether a second one or more infection criteria are satisfied by the temperature data and impedance data generated by the implantable medical device during a second time interval subsequent to the first time interval, wherein the second one or more infection criteria include at least one criterion indicative of increased impedance; and output, based on satisfaction of the first and second infection criteria, an indication of infection.

In another example, a method comprises determining whether a first one or more infection criteria are satisfied by temperature data and impedance data generated by an implantable medical device during a first time interval, wherein the temperature data and impedance data are associated with temperature and impedance of a patient proximate to the implantable medical device, and wherein the first one or more infection criteria include at least one criterion indicative of decreased impedance, determining whether a second one or more infection criteria are satisfied by the temperature data and impedance data generated by the implantable medical device during a second time interval subsequent to the first time interval, wherein the second one or more infection criteria include at least one criterion indicative of increased impedance, and outputting, based on satisfaction of the first and second infection criteria, an indication of infection.

The disclosure also provides means for performing any of the techniques described herein, as well as non-transitory computer-readable media comprising instructions that cause a programmable processor to perform any of the techniques described herein. MOM The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 is a conceptual diagram illustrating an example medical system, including an example implantable medical device (IMD), in conjunction with a patient.

FIG. 2 is a conceptual diagram further illustrating the example IMD of FIG. 1 in conjunction with a heart of the patient.

FIG. 3 is functional block diagram illustrating an example configuration of the IMD of FIG. 1.

FIG. 4 is a functional block diagram illustrating an example configuration of the external computing device of FIG. 1.

FIG. 5 is a block diagram illustrating an example system that includes an access point, a network, external computing devices, such as a server, and one or more other computing devices, which may be coupled to the IMD and external computing device of FIGS. 1-4.

FIG. 6 is a conceptual graph illustrating temperature data and impedance data collected over time by an IMD in accordance with one or more techniques disclosed herein.

FIG. 7 is a flow diagram illustrating an example operation for detecting infection based whether temperature data and impedance data collected by an IMD satisfy one or more first criteria and one or more second criteria.

FIG. 8 is a flow diagram illustrating an example operation for determining whether temperature data and impedance data collected by an IMD satisfy a first one or more infection detection criteria.

FIG. 9 is a flow diagram illustrating an example operation for determining whether temperature data and impedance data collected by an IMD satisfy a second one or more infection detection criteria.

Like reference characters denote like elements throughout the description and figures.

### DETAILED DESCRIPTION

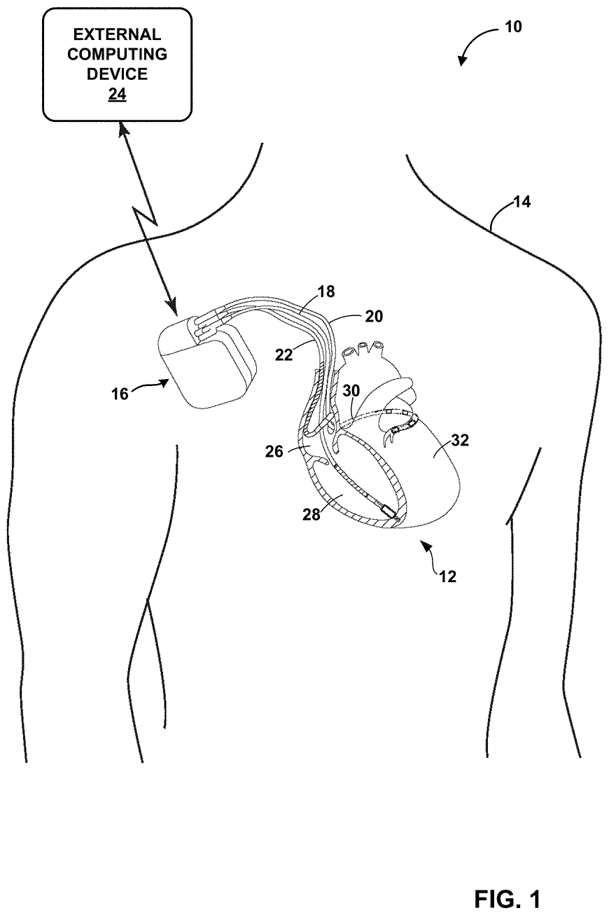

FIG. 1 is a block diagram illustrating system 10 which may be used to detect infection in patient 14, e.g., an infection of an implantation pocket for an implantable medical device (IMD) 16 of system 10, in accordance with the techniques of the disclosure. As illustrated by example system 10 in FIG. 1, IMD 16 may, in some examples, be an implantable cardiac pacemaker, implantable cardioverter/defibrillator (ICD), or pacemaker/cardioverter/defibrillator, for example. While primarily described herein with respect to an implantable cardiac pacemaker, the techniques of this disclosure may be used with any implantable medical device configured to sense temperature and impedance of patient 14. In some examples, IMD 16 may be an implantable monitor, e.g., an insertable cardiac monitor, such as the LINQ II™ commercially available from Medtronic, Inc., of Minneapolis, Minn., an implantable neurostimulator, or an implantable drug pump.

IMD 16 is connected to leads 18, 20 and 22 and is communicatively coupled to external computing device 24. IMD 16 senses electrical signals attendant to the depolarization and repolarization of heart 12, e.g., a cardiac electrogram (EGM), via electrodes on one or more leads 18, 20 and 22 or the housing of IMD 16. IMD 16 may also deliver therapy in the form of electrical signals to heart 12 via electrodes located on one or more leads 18, 20 and 22 or a housing of IMD 16. The therapy may be pacing, cardioversion and/or defibrillation pulses. IMD 16 may monitor EGM signals collected by electrodes on leads 18, 20 or 22, and based on the EGM signal, diagnose, and treat cardiac episodes.

Leads 18, 20, 22 extend into the heart 12 of patient 14 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12. Although IMD 16 is coupled to three leads 18, 20, 22, in the example of FIG. 1, in some examples system 10 may include any suitable number of leads coupled to IMD 16, and each of the leads may extend to any location within or proximate to a heart or in the chest of patient 14.

In the example illustrated by FIG. 1, system 10 includes an external computing device 24 configured to communicate wirelessly with IMD 16. In some examples, external computing device 24 takes the form of a handheld computing device, computer workstation, networked computing device, smartphone, tablet, laptop computer or external programmer that includes a user interface for presenting information to and receiving input from a user. A user, such as a physician, other clinician or caregiver, or patient 14, may interact with external computing device 24 to retrieve physiological or diagnostic information from IMD 16. A user may also interact with external computing device 24 to program IMD 16, e.g., select values for operational parameters of the IMD. External computing device 24 may include processing circuitry configured to evaluate EGM, temperature, impedance, activity or other physiological data transmitted from IMD 16 to external computing device 24.

IMD 16 and external computing device 24 may communicate via wireless communication using any techniques known in the art. Examples of communication techniques may include, for example, communication according to a personal area network technology, such as the Bluetooth ® or BLE protocols, Near Field Communication (NFC) protocols, radio frequency (RF) communication protocols, WI-FI™ protocols, or other proprietary or non-proprietary wireless communication schemes. Other communication techniques are also contemplated. External computing device 24 may also communicate with one or more other external computing devices using several known communication techniques, both wired and wireless.

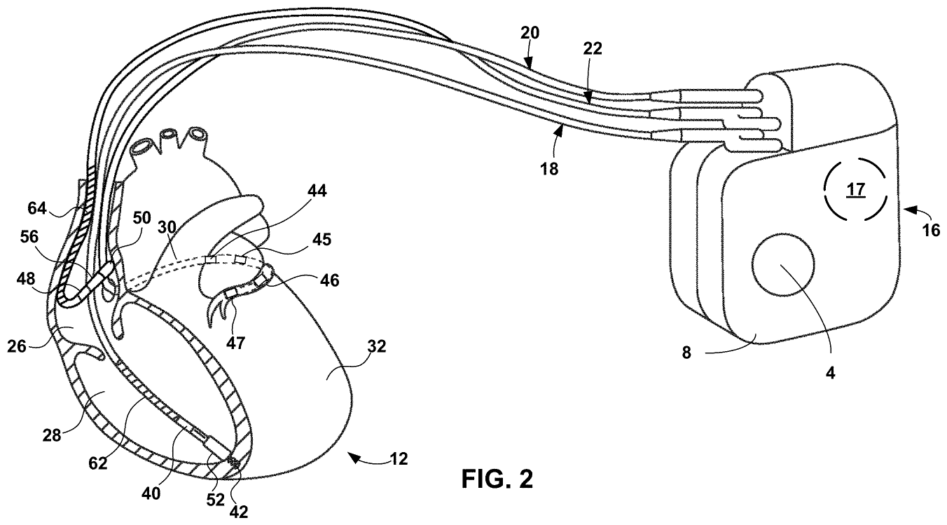

FIG. 2 is a conceptual diagram illustrating IMD 16 and leads 18, 20 and 22 of system 10 in greater detail in conjunction with heart 12. In the illustrated example, bipolar electrodes 40 and 42 are located adjacent to a distal end of lead 18, and bipolar electrodes 48 and 50 are located adjacent to a distal end of lead 22. In addition, four electrodes 44, 45, 46 and 47 are located adjacent to a distal end of lead 20. Lead 20 may be referred to as a quadrapolar LV lead. In other examples, lead 20 may include more or fewer electrodes. In some examples, LV lead 20 comprises segmented electrodes, e.g., in which each of a plurality of longitudinal electrode positions of the lead, such as the positions of electrodes 44, 45, 46 and 47, includes a plurality of discrete electrodes arranged at respective circumferential positions around the circumference of lead.

In the illustrated example, electrodes 40 and 44-48 take the form of ring electrodes, and electrodes 42 and 50 may take the form of extendable helix tip electrodes mounted retractably within insulative electrode heads 52 and 56, respectively. Leads 18 and 22 also include elongated electrodes 62 and 64, respectively, which may take the form of a coil. In some examples, each of electrodes 40, 42, 44-48, 50, 62, and 64 is electrically coupled to a respective conductor within the lead body of its associated lead 18, 20, 22 and thereby coupled to circuitry within IMD 16.

In some examples, IMD 16 includes one or more housing electrodes, such as housing electrode 4 illustrated in FIG. 2, which may be formed integrally with an outer surface of hermetically-sealed housing 8 of IMD 16 or otherwise coupled to housing 8. In some examples, housing electrode 4 is defined by an uninsulated portion of an outward facing portion of housing 8 of IMD 16. Other divisions between insulated and uninsulated portions of housing 8 may be employed to define two or more housing electrodes. In some examples, a housing electrode comprises substantially all of housing 8.

IMD 16 senses electrical signals attendant to the depolarization and repolarization of heart 12 via electrodes 4, 40, 42, 44-48, 50, 62, and 64. IMD 16 may sense such electrical signals via any bipolar combination of electrodes 40, 42, 44-48, 50, 62, and 64. Furthermore, any of the electrodes 40, 42, 44-48, 50, 62, and 64 may be used for unipolar sensing in combination with housing electrode 4.

The illustrated numbers and configurations of leads 18, 20 and 22 and electrodes are merely examples. Other configurations, i.e., number and position of leads and electrodes, are possible. In some examples, system 10 may include an additional lead or lead segment having one or more electrodes positioned at different locations in the cardiovascular system for sensing and/or delivering therapy to patient 14. For example, instead of or in addition to intracardiac leads 18, 20 and 22, system 10 may include one or more epicardial or extravascular (e.g., subcutaneous or substernal) leads not positioned within heart 12.

Processing circuitry of system 10, e.g., of IMD 16, external computing device 24, and/or of one or more other computing devices, may be configured to perform the example techniques of this disclosure for determining whether to provide an indication of infection, e.g., pocket infection, based on temperature data and impedance data generated by IMD 16. The temperature data and impedance data may reflect temperature and impedance of patient 14 proximate to the implantation site of IMD 16. For example, IMD 16 may measure temperature with a temperature sensing device on or within housing 8 of IMD 16, and impedance using an electrode of housing 8, such as electrode 4. In this manner, features of the temperature data and impedance data generated by IMD 16 may be indicative of the condition of tissue of patient 14 proximate to IMD 16, and this the pocket in which IMD 16 is implanted.

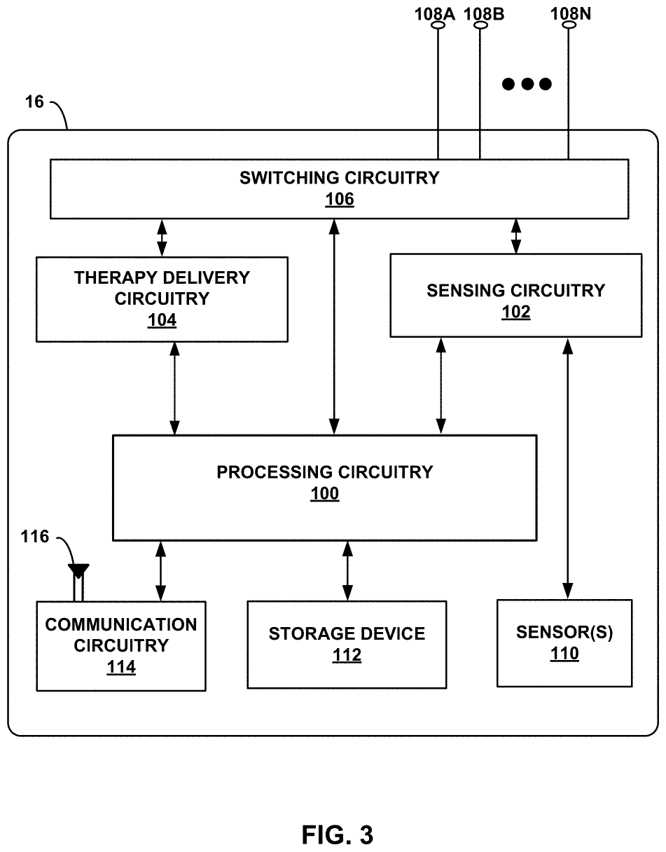

FIG. 3 is a functional block diagram illustrating an example configuration of IMD 16 of FIGS. 1 and 2 in accordance with one or more techniques described herein. In the illustrated example, IMD 16 includes processing circuitry 100. IMD 16 also include sensing circuitry 102 and therapy delivery circuitry 104 coupled to processing circuitry 100, and selectively coupled to one or more of electrodes 108A-108N (collectively, “electrodes 108”) via switching circuitry 106. Electrodes 108 may correspond to electrodes 4, 40, 42, 44-48, 50, 62, and 64 illustrated in FIGS. 1 and 2.

Processing circuitry 100 may include fixed function circuitry and/or programmable processing circuitry. Processing circuitry 100 may include any one or more of a microprocessor, a graphical processing unit (GPU), a tensor processing unit (TPU), a controller, a digital signal processor (DSP), an application specific integrated circuit (ASIC), a field-programmable gate array (FPGA), or equivalent discrete or analog logic circuitry. In some examples, processing circuitry 100 may include multiple components, such as any combination of one or more microprocessors, one or more GPUs, one or more TPUs, one or more controllers, one or more DSPs, one or more ASICs, or one or more FPGAs, as well as other discrete or integrated logic circuitry. The functions attributed to processing circuitry 100 herein may be embodied as software, firmware, hardware or any combination thereof

Sensing circuitry 102 may monitor the electrical activity of heart via selected combinations of electrodes 108, e.g., to detect cardiac depolarizations. Processing circuitry 100 may determine heart rate and detect arrhythmias based on the detection of depolarizations by sensing circuitry 102. Sensing circuitry 102 may include an analog-to-digital converter to digitize the cardiac electrical signals, e.g., cardiac EGM, and processing circuitry 100 may analyze the digitized signals, e.g., to detect arrhythmias. Sensing circuitry 102 may also measure impedance via selected combinations of electrodes 108.

Sensing circuitry 102 may also receive signals from sensor(s) 110, which may include one or more temperature sensors, accelerometers, pressure sensors, and/or optical sensors, as examples. Sensor(s) 110 may include one or more temperature sensing devices. Any suitable sensor(s) 110 may be used to detect temperature or changes in temperature. In some examples, sensor(s) 110 may include a thermocouple, a thermistor, a junction-based thermal sensor, a thermopile, a fiber optic detector, an acoustic temperature sensor, a quartz or other resonant temperature sensor, a thermo-mechanical temperature sensor, a thin film resistive element, etc.

In some examples, processing circuitry 100 and/or sensing circuitry 102 may implement or include one or more filters and amplifiers for filtering and amplifying signals received from sensor(s) 110 and/or electrodes 108. For example, processing circuitry 100 and/or sensing circuitry 102 may implement one or more low-pass filters having various cutoff frequencies predefined to apply to temperature values obtained from sensor(s) 110 and impedance values measured via electrode 108. In some examples, processing circuitry 100 and/or sensing circuitry 102 may include circuitry configured to digitally filter measured temperature values and impedance values using one or more cutoff frequencies, or otherwise using one or more different filtering processes to achieve different degrees of smoothing of a series of temperature values and/or impedance values. For example, processing circuitry 100 may include circuitry configured to average temperature values measured within respective time intervals, thereby smoothening the series of temperature values and reducing the possibility of false-positive infection detections.

Processing circuitry 100 may cause sensing circuitry 102 to periodically measure temperature and impedance values, e.g., according to a schedule. The measurements may occur at a sampling rate, e.g., 0.1 Hz or any other sampling rate, for a period of time, e.g., a number of consecutive minutes or hours, and may occur daily or multiple times per day. As one example, processing circuitry 100 may cause sensing circuitry 102 to measure temperature and impedance values during a predetermined two hour period each day. In some examples, since activity and/or posture may affect temperature and impedance of patient 14, the measurements may be scheduled to occur when patient 14 is expected to be inactive and in a consistent, e.g., lying, posture, such as at night.

In some examples, sensor(s) 110 may include an accelerometer or other sensor of patient motion, activity or posture. In some examples, processing circuitry 100 may control sensing circuitry 102 to measure temperature and impedance based on the patient activity and/or posture. For example, processing circuitry 100 may control sensing circuitry to measure temperature and impedance when patient 14 has an activity level below a threshold and is in a desired posture, or to not measure temperature and impedance when patient 14 has an activity level above a threshold and/or is not in a desired posture. In some examples, processing circuitry 100 may discard temperature data and impedance data generated when patient 14 has an activity level above a threshold and/or is not in a desired posture. In some examples, processing circuitry 100 may additionally or alternatively control measurements and/or discard data based on heart rate and/or ambient temperature.

In various examples, processing circuitry 100 may perform one, all, or any combination of the plurality of infection detection techniques discussed in greater detail below. In performing the infection detection techniques, IMD 16 may generate an alert upon determining patient 14 is likely to have an infection, such as a device pocket infection. For example, IMD 16 may provide an audible or tactile alert in the form of a beeping noise or a vibrational pattern. In some examples, IMD 16 may send an alert signal to external computing device 24 or another computing device or system that causes the device or system to provide an audible, visual, or tactile alert to patient 14, or a clinician or caregiver.

Sensing circuitry 102 may provide one or more temperature and impedance values to processing circuitry 100 for analysis, e.g., for analysis to identify a possible infection according to the techniques of this disclosure. In some examples, processing circuitry 100 may store the temperature and impedance values to storage device 112 as temperature and impedance data. Processing circuitry 100 of IMD 16, and/or processing circuitry of another device that retrieves data from IMD 16, may analyze the temperature and impedance data to detect infection according to the techniques of this disclosure.

In the example illustrated by FIG. 3, IMD 16 includes communication circuitry 114, which may include any suitable hardware, firmware, software or any combination thereof for communicating with another device, such as external computing device 24, another networked computing device, or another IMD or sensor. Under the control of processing circuitry 100, communication circuitry 114 may receive downlink telemetry from, as well as send uplink telemetry to external computing device 24 or another device with the aid of an internal or external antenna, e.g., antenna 116. In addition, processing circuitry 100 may communicate with a networked computing device via an external device (e.g., external computing device 24) and a computer network, such as the Medtronic CareLink® Network. Antenna 116 and communication circuitry 114 may be configured to transmit and/or receive signals via inductive coupling, electromagnetic coupling, near-field communications, RF communication, Bluetooth®, WI-FI™, or other proprietary or non-proprietary wireless communication schemes.

In some examples, processing circuitry 100 may send temperature data and impedance data to external computing device 24 or another networked computing device via communication circuitry 114. The computing device may perform the techniques for analyzing the temperature and impedance data to detect infection described herein. Alternatively, processing circuitry 100 may perform the processing techniques and transmit the processed data and/or indications of whether infection is detected to external computing device 24 or another computing device for reporting purposes, e.g., for providing an alert to patient 14 or another user.

In some examples, storage device 112 includes computer-readable instructions that, when executed by processing circuitry 100, cause IMD 16 and processing circuitry 100 to perform various functions attributed to IMD 16 and processing circuitry 100 herein. Storage device 112 may include any volatile, non-volatile, magnetic, optical, or electrical media. For example, storage device 112 may include random access memory (RAM), read-only memory (ROM), non-volatile RAM (NVRAM), electrically-erasable programmable ROM (EEPROM), erasable programmable ROM (EPROM), flash memory, or any other digital media. Storage device 112 may store, as examples, programmed values for one or more operational parameters of IMD 16 and/or data collected by IMD 16 for analysis by processing circuitry 100 and/or transmission to another device using communication circuitry 114. Data stored by storage device 112 and transmitted by communication circuitry 114 to one or more other devices may include temperature data and impedance data.

As shown in FIG. 3, IMD 16 includes therapy delivery circuitry 104; however, the inclusion of therapy delivery circuitry 104 is optional. In examples, where IMD 16 includes therapy delivery circuitry 104, processing circuitry 100 may control therapy delivery circuitry 104 to deliver therapy to heart 12 via selected combinations of electrodes 108. For example, processing circuitry 100 may control therapy delivery circuitry 104 to deliver electrical pulses with amplitudes, pulse widths, frequency, or electrode polarities specified by the therapy parameters stored in storage device 112. In this manner, therapy delivery circuitry 104 may deliver pacing pulses (e.g., overdrive pacing pulses, anti-tachycardia pacing pulses, bradycardia pacing pulses, cardiac resynchronization therapy, or post-shock pacing therapy) to heart 12 via one or more combinations of electrodes 108. In some examples, processing circuitry 100 controls therapy delivery circuitry 104 to deliver anti-tachyarrhythmia shocks via selected combinations of electrodes 108, e.g., including electrodes 62 and 64 illustrated in FIG. 2). In some examples, therapy delivery circuitry 104 includes a charging circuit, one or more pulse generators, capacitors, transformers, switching modules, and/or other components capable of generating and/or storing energy to deliver as pacing therapy, anti-tachyarrhythmia shock therapy, or other therapy.

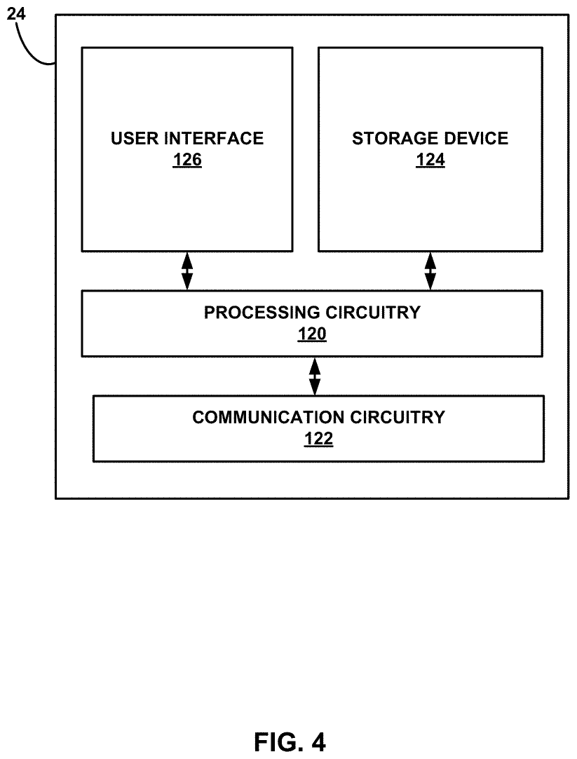

FIG. 4 is a block diagram illustrating an example configuration of external computing device 24. In the example of FIG. 4, external computing device 24 includes processing circuitry 120, communication circuitry 122, storage device 124, and user interface 126.

Processing circuitry 120 may include one or more processors that are configured to implement functionality and/or process instructions for execution within external computing device 24. For example, processing circuitry 120 may be capable of processing instructions stored in storage device 124. Processing circuitry 120 may include, for example, microprocessors, GPUs, TPUs, DSPs, ASICs, FPGAs, or equivalent discrete or integrated logic circuitry, or a combination of any of the foregoing devices or circuitry. Accordingly, processing circuitry 120 may include any suitable structure, whether in hardware, software, firmware, or any combination thereof, to perform the functions ascribed herein to processing circuitry 120.

Communication circuitry 122 may include any suitable hardware, firmware, software or any combination thereof for communicating with another device, such as IMD 16. Under the control of processing circuitry 120, communication circuitry 122 may receive downlink telemetry from, as well as send uplink telemetry to, IMD 16, or another device. Communication circuitry 122 may be configured to transmit or receive signals via inductive coupling, electromagnetic coupling, Near Field Communication (NFC), RF communication, Bluetooth®, WI-FI™, or other proprietary or non-proprietary wireless communication schemes. Communication circuitry 122 may also be configured to communicate with devices other than IMD 16 via any of a variety of forms of wired and/or wireless communication and/or network protocols.

Storage device 114 may be configured to store information within external computing device 24 during operation. Storage device 124 may include a computer-readable storage medium or computer-readable storage device. In some examples, storage device 124 includes one or more of a short-term memory or a long-term memory. Storage device 124 may include, for example, RAM, DRAM, SRAM, magnetic discs, optical discs, flash memories, or forms of EPROM or EEPROM. In some examples, storage device 124 is used to store data indicative of instructions for execution by processing circuitry 120. Storage device 124 may be used by software or applications running on external computing device 24 to temporarily store information during program execution. Storage device 124 may also store temperature data and impedance data retrieved from IMD 16. Processing circuitry 120 may implement any of the techniques described herein to analyze temperature data, impedance data and activity data received from IMD 16 to detect infection. Using the temperature and impedance analysis techniques disclosed herein, processing circuitry 120 may determine an infection status of patient 14 and/or generate an alert via user interface 126 based on the infection status.

A user, such as a clinician or patient 14, may interact with external computing device 24 through user interface 126. User interface 126 includes a display (not shown), such as a liquid crystal display (LCD) or a light emitting diode (LED) display or other type of screen, with which processing circuitry 120 may present information related to IMD 16, e.g., cardiac EGMs, temperature and impedance data, and indications of infection. In addition, user interface 126 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 120 of external computing device 24 and provide input. In other examples, user interface 126 also includes audio circuitry for providing audible notifications, instructions, or other sounds to the user, receiving voice commands from the user, or both.

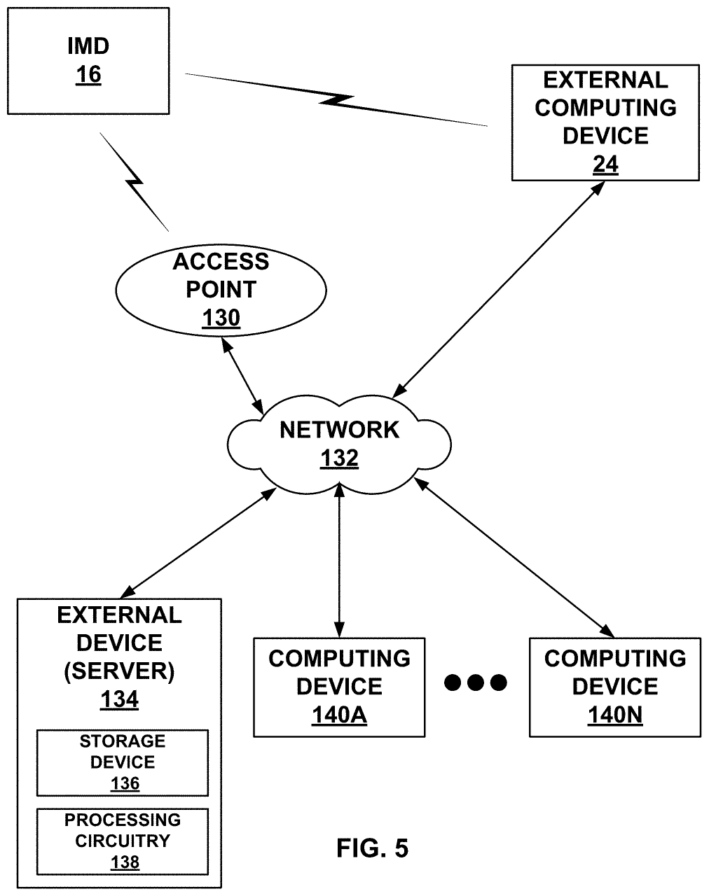

FIG. 5 is a block diagram illustrating an example system that includes an access point 130, a network 132, external computing devices, such as a server 134, and one or more other computing devices 140A-140N (collectively, “computing devices 140”), which may be coupled to IMD 16 and external computing device 24 via network 132, in accordance with one or more techniques described herein. In this example, IMD 16 may use communication circuitry 114 to communicate with external computing device 24 via a first wireless connection, and to communicate with an access point 130 via a second wireless connection. In the example of FIG. 5, access point 130, external computing device 24, server 134, and computing devices 130 are interconnected and may communicate with each other through network 132. Network 132 may comprise a local area network, wide area network, or global network, such as the Internet. The system of FIG. 5 may be implemented, in some aspects, with general network technology and functionality similar to that provided by the Medtronic CareLink® Network.

Access point 130 may include a device that connects to network 92 via any of a variety of connections, including wired or wireless connections. In some examples, access point 130 may be a user device, such as a tablet or smartphone, that may be co-located with the patient. IMD 16 may be configured to transmit data, such as temperature data, impedance data, and indications of infection, to access point 130. Access point 130 may then communicate the retrieved data to server 134 via network 132.

In some cases, server 134 may be configured to provide a secure storage site for data that has been collected from IMD 16 and/or external computing device 24. In some cases, server 134 may assemble data in web pages or other documents for viewing by trained professionals, such as clinicians, via computing devices 140. One or more aspects of the illustrated system of FIG. 5 may be implemented with general network technology and functionality, which may be similar to that provided by the Medtronic CareLink® Network.

In some examples, server 134 may analyze temperature data, impedance data and activity data received from IMD 16 via network 132 to identify an infection status of patient 14 using any of the techniques described herein. Server 134 may provide alerts relating to an infection status of patient 14 via network 132 to patient 14 via access point 130, or to one or more clinicians via computing devices 140. In examples such as those described above in which IMD 16 and/or external computing device 24 analyze temperature and impedance, server 134 may receive an alert from IMD 16 or external computing device 24 via network 132, and provide alerts to one or more clinicians via computing devices 140. In some examples, server 134 may generate web-pages to provide alerts and information regarding the infection status of patient 4, and may comprise a memory to store alerts and diagnostic or physiological parameter information for a plurality of patients.

In some examples, one or more of computing devices 140 may be a tablet or other smart device located with a clinician, by which the clinician may program, receive alerts from, and/or interrogate IMD 16. For example, the clinician may access data collected by IMD 16 through a computing device 140, such as when patient 14 is in between clinician visits, to check on a status of a medical condition. In some examples, the clinician may enter instructions for a medical intervention for patient 14 into an application executed by computing device 140, such as based on a status of a patient condition determined by IMD 16, external computing device 24, server 134, or any combination thereof, or based on other patient data known to the clinician. Device 140 then may transmit the instructions for medical intervention to another of computing devices 140 located with patient 14 or a caregiver of patient 14. For example, such instructions for medical intervention may include an instruction to change a drug dosage, timing, or selection, to schedule a visit with the clinician, or to seek medical attention. In further examples, a computing device 140 may generate an alert to patient 14 based on a status of a medical condition of patient 14, which may enable patient 14 proactively to seek medical attention prior to receiving instructions for a medical intervention. In this manner, patient 14 may be empowered to take action, as needed, to address his or her medical status, which may help improve clinical outcomes for patient 14.

In the example illustrated by FIG. 5, server 134 includes a storage device 136, e.g., to store data retrieved from IMD 16, and processing circuitry 138. Although not illustrated in FIG. 5 computing devices 140 may similarly include a storage device and processing circuitry. Processing circuitry 138 may include one or more processors that are configured to implement functionality and/or process instructions for execution within server 134. For example, processing circuitry 138 may be capable of processing instructions stored in storage device 136 (e.g., stored in memory). Processing circuitry 138 may include, for example, microprocessors, GPUs, TPUs, DSPs, ASICs, FPGAs, or equivalent discrete or integrated logic circuitry, or a combination of any of the foregoing devices or circuitry. Accordingly, processing circuitry 138 may include any suitable structure, whether in hardware, software, firmware, or any combination thereof, to perform the functions ascribed herein to processing circuitry 138. Processing circuitry 138 of server 134 and/or the processing circuitry of computing devices 140 may implement any of the techniques described herein to analyze temperature data and impedance data received from IMD 16 to determine an infection status of patient 14 (e.g., to detect a device pocket infection).

Storage device 136 may include a computer-readable storage medium or computer-readable storage device. In some examples, storage device 136 includes one or more of a short-term memory or a long-term memory. Storage device 136 may include, for example, RAM, DRAM, SRAM, magnetic discs, optical discs, flash memories, or forms of EPROM or EEPROM. In some examples, storage device 136 is used to store data indicative of instructions for execution by processing circuitry 138.

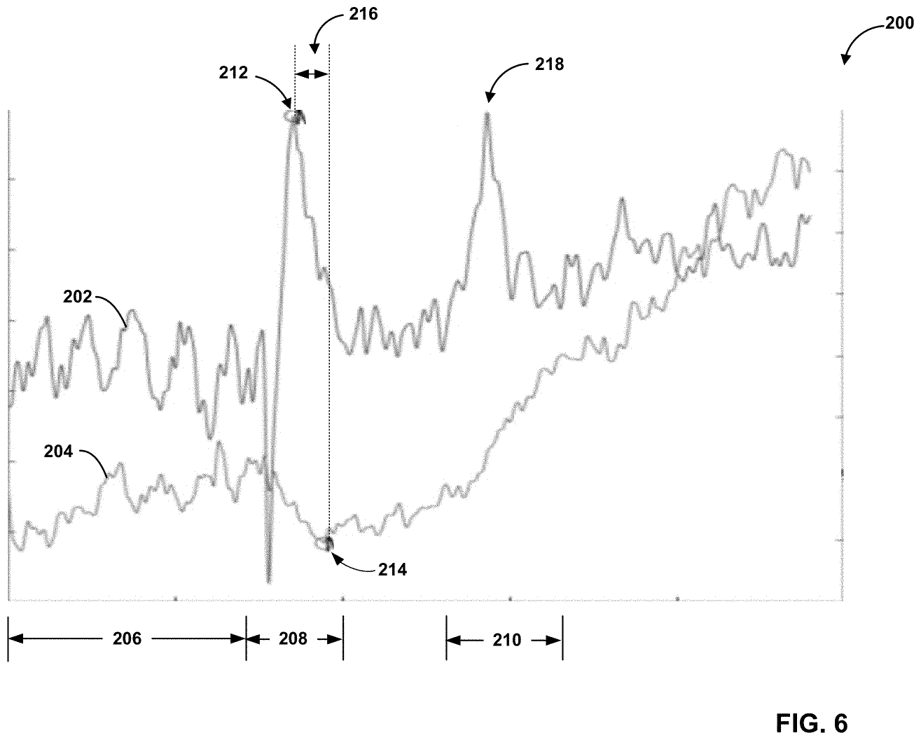

FIG. 6 is a conceptual graph 200 illustrating example temperature data 202 and impedance data 204 that may be collected over time by IMD 16 in accordance with one or more techniques disclosed herein. Prior to an infection, IMD 16 collects baseline temperature data and baseline impedance data during time interval 206. An infection occurs during time interval 208. Time interval 206 that includes the baseline data may have a fixed, predetermined length, or a variable length. Processing circuitry implementing the techniques of this disclosure, e.g., processing circuitry 100 of IMD 16, processing circuitry 120 of external computing device 24, or processing circuitry 138 of server 134, may process temperature and impedance data on a segment-by-segment or interval-by-interval basis. Time interval 206 may include a predetermined number of segments preceding the current segments under consideration, or all segments that preceded the current segments. In some examples, each segment includes two hours of temperature and impedance data sampled at 0.1 Hz.

As illustrated by FIG. 6, temperature proximate IMD 16 increases during interval 208 during which a pocket infection occurs. The pocket infection is associated with increased blood perfusion at the infection site, resulting in decreased impedance during time interval 208, as illustrated by the example of FIG. 6. Processing circuitry implementing the infection techniques of this disclosure may determine whether a first one or more infection criteria are satisfied by temperature data 202 and impedance data 204 generated by IMD 16 during time interval 208. The first one or more infection criteria include at least one criterion indicative of decreased impedance.

For example, to determine whether at least one criterion indicative of decreased impedance is satisfied, the processing circuitry may be configured to compare impedance data 204 during time interval 208 to baseline impedance data 204 during time interval 206. In some examples, the processing circuitry is configured to determine an average, e.g., mean, median, or other mid-based statistic, of both the impedance data 204 during time interval 208 and the baseline impedance data 204 during time interval 206, and compare the averages. In some examples, the processing circuitry may determine whether a difference or ratio between the averages satisfies a criterion, e.g., exceeds a threshold.

In some examples, the processing circuitry is configured to compare the impedance data 204 generated during time interval 208 to the baseline impedance data 204 using a short-term moving average to long-term moving average comparison technique. For example, the processing circuitry may determine a short-term moving average of impedance data 204 including impedance data 204 generated during time interval 208, and determine a long-term moving average of impedance data 204 comprising the baseline impedance data from time interval 206. In some examples, the short-term moving average may include impedance data 204 from time interval 206 and/or the long-term moving average may include impedance data from time interval 208. However, the long-term moving average will generally include data from further in the past than the short-term moving average. In this manner, the comparison will identify recent changes in the trend of the data, e.g., recent decreases in impedance. The processing circuitry may accumulate differences between the short-term moving average and the long-term moving average, and an increase in magnitude of the accumulated differences may indicate deviation of recent values from the baseline in a consistent positive or negative direction. In some examples, processing circuitry may compare the accumulated value to a threshold to determine whether a criterion, e.g., indicative of decreased impedance, is satisfied.

To determine whether the first one or more infection criteria are satisfied, the processing circuitry may be configured to determine a maximum temperature 212 and/or a minimum impedance 214 during time interval 208. The processing circuitry may determine whether the first one or more infection criteria are satisfied by comparing one or both of the maximum temperature 212 or minimum impedance 214 to a respective threshold temperature or impedance. In some examples, the processing circuitry may determine a difference 216 between a time of the maximum temperature and a time of the minimum impedance. The processing circuitry may determine whether the first one or more infection criteria are satisfied by comparing the difference to a threshold difference, e.g., to determine whether the difference is no greater than the threshold difference. The start of a pocket infection may result in changes in temperature and impedance of tissue proximate to the pocket that are reflected in a maximum of temperature and a minimum of impedance occurring within a certain time proximity. In some examples, the processing circuitry may output an indication of infection based on satisfaction of the first infection detection criteria, which may result in delivery of an alert via IMD 16, computing device 24, and/or a computing device 140.

The temperature data and impedance data may include features indicative of infection occurring a number of days, e.g., three to six days, after the initial infection. Time interval 210 may extend or include data generated at least three days after time interval 208. For example, temperature data 202 during time interval 210 may remain increased relative to baseline temperature data 202 during time interval 206, and may include a second peak 218. Furthermore, impedance data 204 during time interval 210 may be increased, e.g., relative to baseline impedance data 204 during time interval 206 and/or impedance data 204 during time interval 208. Temperature data 202 and impedance data 204 may exhibit a sustained rise after the initial infection, e.g., three to six days after the initial infection, such as during time interval 210.

Processing circuitry implementing the infection techniques of this disclosure, e.g., processing circuitry 100 of IMD 16, processing circuitry 120 of external computing device 24, or processing circuitry 138 of server 134, may determine whether a second one or more infection criteria are satisfied by temperature data 202 and impedance data 204 generated during time interval 210 subsequent to time interval 208. In some examples, the processing circuitry determines whether the second one or more infection criteria are satisfied in response to satisfaction of the first one or more criteria. The second one or more infection criteria may include at least one criterion indicative of increased impedance.

For example, to determine whether the second one or more infection criteria are satisfied, the processing circuitry may be configured to compare temperature data 202 and/or impedance data 204 generated during time interval 210 to, e.g., baseline data from time interval 206 and/or data from time interval 208. The comparison may include comparing averages and/or comparing short-term and long-term moving averages, as described above. The second one or more infection criteria may include respective threshold differences and/or threshold accumulated values indicating that temperature and impedance have increased relative to their baselines. In some examples, to determine whether the second one or more infection criteria are satisfied, the processing circuitry is configured to determine whether a peak 218 occurs in temperature data 202 generated during time interval 210.

Satisfaction of the second one or more infection detection criteria may confirm an indication of infection by satisfaction of the first one or more infection detection criteria. In some examples, the processing circuitry may output an indication of infection based on satisfaction of the second infection detection criteria, which may result in delivery of an alert via IMD 16, computing device 24, and/or a computing device 140. In some examples, the processing circuitry delivers a single alert based on satisfaction of the first one or more infection detection criteria and the second one or more infection detection criteria.

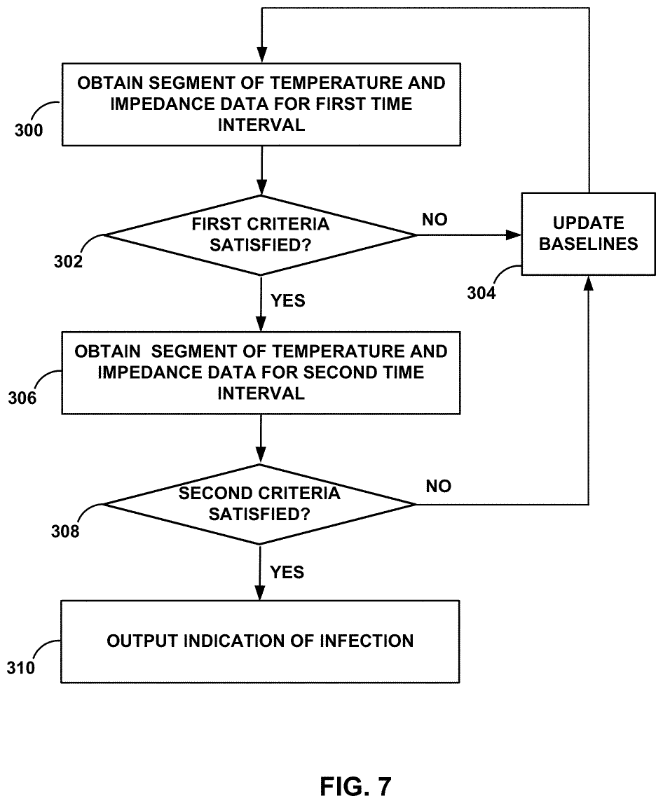

FIG. 7 is a flow diagram illustrating an example operation for detecting infection based whether temperature data and impedance data collected by IMD 16 satisfy one or more first criteria and one or more second criteria. Although described as being performed by IMD 16, the example method of FIG. 7 may be performed by any one or more of IMD 16, external computing device 24, or server 134, e.g., by the processing circuitry of any one or more of these devices. For example, IMD 16 may transmit temperature data and impedance data to external computing device 24 and/or server 134, with external computing device 24 and/or server 134 performing various other techniques of this disclosure.

According to the example technique of FIG. 7, processing circuitry 100 of IMD 16 obtains, e.g., from storage device 112 a segment of temperature data 202 and impedance data 204 generated by IMD 16 during a first time interval 208 (300). Processing circuitry 100 determines whether the temperature data and impedance data during first time interval 208 satisfy a first one or more infection detection criteria, e.g., as described above with respect to FIG. 6 (302). If the first one or more infection criteria are not satisfied (NO of 302), processing circuitry 100 updates the baseline temperature data and baseline impedance data (304) and obtains a next segment of temperature data and impedance data as the first time interval (300).

If the first one or more infection criteria are satisfied (YES of 302), processing circuitry 100 obtains a segment of temperature data 202 and impedance data 204 generated by IMD 16 during a second time interval 210 (306). As discussed above, second time interval 210 may occur (e.g., begin or be centered at a time) a number of days (e.g., three to six) after first time interval 208. Processing circuitry 100 determines whether the temperature data and impedance data during second time interval 210 satisfy a second one or more infection detection criteria, e.g., as described above with respect to FIG. 6 (308). If the second one or more infection criteria are not satisfied (NO of 308), processing circuitry 100 updates the baseline temperature data and baseline impedance data (304) and obtains a next segment of temperature data and impedance data as the first time interval (300). If the second one or more infection criteria are satisfied (YES of 308), processing circuitry 100 outputs an indication of infection, e.g., to external computing device 24 or server 134 (310).

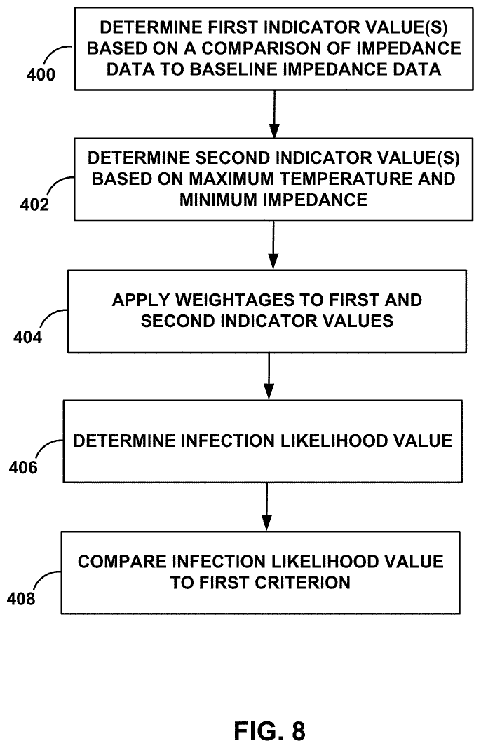

FIG. 8 is a flow diagram illustrating an example operation for determining whether temperature data 202 and impedance data 204 collected by IMD 16 during first time interval 208 satisfy a first one or more infection detection criteria. The example operation of FIG. 8 may be one example of operation 302 of FIG. 7. Although described as being performed by IMD 16, the example method of FIG. 8 may be performed by any one or more of IMD 16, external computing device 24, or server 134, e.g., by the processing circuitry of any one or more of these devices. For example, IMD 16 may transmit temperature data and impedance data to external computing device 24 and/or server 134, with external computing device 24 and/or server 134 performing various other techniques of this disclosure.

According to the example of FIG. 8, processing circuitry 100 determines one or more first indicator values based on one or more comparisons of impedance data 204 from interval 208 to baseline impedance data from interval 206 (400). The comparisons may include comparing averages and/or comparing short-term and long-term moving averages, as described above. Processing circuitry 100 further determines one or more second indicator values based on a maximum 212 of temperature data 202 and a minimum 214 of impedance data 204 during interval 206 (402). In some examples, processing circuitry 100 may determine a second indicator value based on a difference 216 between a time of the maximum temperature and a time of the minimum impedance.

Processing circuitry 100 applies respective weightages to the one or more first indicator values and the one or more second indicator values (404). In some examples, processing circuity 100 applies a greater weightage value to the one or more second indicator values (e.g., 100%) than to the one or more first indicator values (e.g., 83%). Processing circuitry 100 further determines an infection likelihood value based on the application of a first weightage to the first indicator value(s) and a second weightage to the second indicator value(s) (406). Processing circuitry 100 compares the infection likelihood value with a first infection detection criterion to determine whether the first infection detection criterion is satisfied (408).

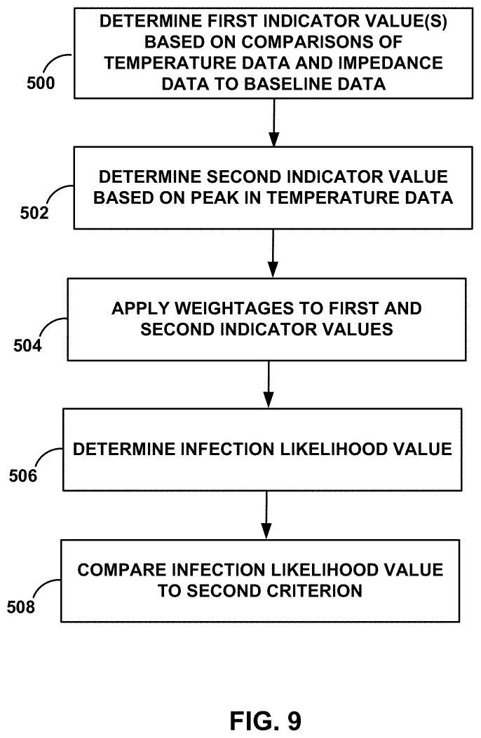

FIG. 9 is a flow diagram illustrating an example operation for determining whether temperature data 202 and impedance data 202 collected by IMD 16 satisfy a second one or more infection detection criteria. The example operation of FIG. 9 may be one example of operation 308 of FIG. 7. Although described as being performed by IMD 16, the example method of FIG. 9 may be performed by any one or more of IMD 16, external computing device 24, or server 134, e.g., by the processing circuitry of any one or more of these devices. For example, IMD 16 may transmit temperature data and impedance data to external computing device 24 and/or server 134, with external computing device 24 and/or server 134 performing various other techniques of this disclosure.

According to the example of FIG. 9, processing circuitry 100 determines one or more first indicator values based on one or more comparisons of temperature data 202 and impedance data 204 from interval 210 to baseline temperature and impedance data from interval 206 (500). The comparisons may include comparing averages and/or comparing short-term and long-term moving averages, as described above. Processing circuitry 100 further determines one or more second indicator values based on identification of a peak 218 of temperature data 202 during interval 210 (502).

Processing circuitry 100 applies respective weightages to the one or more first indicator values and the one or more second indicator values (504). In some examples, processing circuity 100 applies a greater weightage value to the one or more first indicator values (e.g., 100%) than to the one or more first indicator values (e.g., 50%). Processing circuitry 100 further determines an infection likelihood value based on the application of a first weightage to the first indicator value(s) and a second weightage to the second indicator value(s) (506). Processing circuitry 100 compares the infection likelihood value to a second infection detection criterion to determine whether the second infection detection criterion is satisfied (508).

This disclosure includes various examples, such as the following examples.

Example 1: A system includes an implantable medical device configured to generate temperature data and impedance data associated with temperature and impedance of a patient proximate to the implantable medical device; and processing circuitry configured to: determine whether a first one or more infection criteria are satisfied by temperature data and impedance data generated by the implantable medical device during a first time interval, wherein the first one or more infection criteria include at least one criterion indicative of decreased impedance; determine whether a second one or more infection criteria are satisfied by the temperature data and impedance data generated by the implantable medical device during a second time interval subsequent to the first time interval, wherein the second one or more infection criteria include at least one criterion indicative of increased impedance; and output, based on satisfaction of the first and second infection criteria, an indication of infection.

Example 2: The system of example 1, wherein the processing circuitry is configured to determine whether the one or more second infection criteria are satisfied in response to satisfaction of the one or more first infection criteria.

Example 3: The system of example 1 or 2, wherein the second time interval extends at least three days later than the first time interval.

Example 4: The system of any of examples 1 to 3, wherein, to determine whether the first one or more infection criteria are satisfied, the processing circuitry is configured to determine a maximum temperature during the first time interval.

Example 5: The system of any of examples 1 to 4, wherein, to determine whether the at least one criterion indicative of decreased impedance is satisfied, the processing circuitry is configured to determine a minimum impedance during the first time interval. 100821 Example 6: The system of examples 4 and 5, wherein, to determine whether the first one or more infection criteria are satisfied, the processing circuitry is configured to determine a difference between a time of the maximum temperature and a time of the minimum impedance.

Example 7: The system of any of examples 1 to 6, wherein, to determine whether the at least one criterion indicative of decreased impedance is satisfied, the processing circuitry is configured to compare the impedance data generated during the first time interval to baseline impedance data generated by the implantable medical device prior to the first time interval.

Example 8: The system of example 7, wherein, to compare the impedance data generated during the first time interval to baseline impedance data generated by the implantable medical device prior to the first time interval, the processing circuitry is configured to: determine a short-term moving average of impedance data generated by the implantable medical device including the impedance data generated during the first time interval; determine a long-term moving average of impedance data generated by the implantable medical device including the baseline impedance data; and accumulate differences between the short-term moving average and the long-term moving average.

Example 9: The system of any of examples 4 to 6 in combination with example 7 or 8 wherein, to determine whether the first one or more infection criteria are satisfied, the processing circuitry is configured to: determine a first indicator value based on one or more of the maximum temperature, the minimum impedance, or the difference between the time of the maximum temperature and the time of the minimum impedance; determine a second indicator value based on the comparison of the impedance data generated during the first time interval to the baseline impedance data; and determine an infection likelihood value based on application of a first weightage to the first indicator value and a second weightage to the second indicator value, wherein the first weightage is greater than the second weightage.

Example 10: The system of any of examples 1 to 9, wherein, to determine whether the second one or more infection criteria are satisfied, the processing circuitry is configured to determine whether a peak occurs in the temperature data generated during the second time interval.

Example 11: The system of any of examples 1 to 10, wherein, to determine whether the second one or more infection criteria are satisfied, the processing circuitry is configured to compare the temperature data generated during the second time interval to baseline temperature data generated by the implantable medical device before the first time interval.

Example 12: The system of example 11, wherein, to compare the temperature data generated during the second time interval to the baseline temperature data, the processing circuitry is configured to: determine a short-term moving average of temperature data generated by the implantable medical device including temperature data generated during the second time interval; determine a long-term moving average of temperature data generated by the implantable medical device including the baseline temperature data; and accumulate differences between the short-term moving average and the long-term moving average.

Example 13: The system of any of examples 1 to 12, wherein, to determine whether the at least one criterion indicative of increased impedance is satisfied, the processing circuitry is configured to compare the impedance data generated during the second time interval to baseline impedance data generated by the implantable medical device before the first time interval.

Example 14: The system of any of examples 1 to 13, wherein, to compare the impedance data generated during the second time interval to the baseline impedance data, the processing circuitry is configured to: determine a short-term moving average of impedance data generated by the implantable medical device including the impedance data generated during the second time interval; determine a long-term moving average of impedance data generated by the implantable medical device including the baseline impedance data; and accumulate differences between the short-term moving average and the long-term moving average.

Example 15: The system of any of examples 10 to 14, wherein, to determine whether the second one or more infection criteria are satisfied, the processing circuitry is configured to: determine a first indicator value based on one or more of the comparison of the temperature data generated during the second time interval to the baseline temperature data or the comparison of the impedance data generated during the second time interval to the baseline impedance data; determine a second indicator value based on the peak in the temperature data generated during the second time interval; and determine an infection likelihood value based on application of a first weightage to the first indicator value and a second weightage to the second indicator value, wherein the first weightage is greater than the second weightage.

Example 16: The system of any of examples 1 to 15, further includes determine at least one of an activity level or posture of the patient based on a signal from the motion sensor; and discard at least a portion of at least one of the temperature data or the impedance data based on the activity level or posture.

Example 17: The system of any of examples 1 to 16, wherein the indication of infection is an indication of pocket infection.

Example 18: The system of any of examples 1 to 17, wherein the processing circuitry includes processing circuitry of the implantable medical device.

Example 19: A method includes determining whether a first one or more infection criteria are satisfied by temperature data and impedance data generated by an implantable medical device during a first time interval, wherein the temperature data and impedance data are associated with temperature and impedance of a patient proximate to the implantable medical device, and wherein the first one or more infection criteria include at least one criterion indicative of decreased impedance; determining whether a second one or more infection criteria are satisfied by the temperature data and impedance data generated by the implantable medical device during a second time interval subsequent to the first time interval, wherein the second one or more infection criteria include at least one criterion indicative of increased impedance; and outputting, based on satisfaction of the first and second infection criteria, an indication of infection.

Example 20: The method of example 19, wherein determining whether the one or more second infection criteria are satisfied includes determining whether the one or more second infection criteria are satisfied in response to satisfaction of the one or more first infection criteria.

Example 21: The method of example 19 or 20, wherein the second time interval extends at least three days later than the first time interval.

Example 22: The method of any of examples 19 to 21, wherein determining whether the first one or more infection criteria are satisfied includes determining a maximum temperature during the first time interval.

Example 23: The method of any of examples 19 to 22, wherein determining whether the at least one criterion indicative of decreased impedance is satisfied includes determining a minimum impedance during the first time interval.

Example 24: The method of examples 22 and 23, wherein determining whether the first one or more infection criteria are satisfied includes determining a difference between a time of the maximum temperature and a time of the minimum impedance.

Example 25: The method of any of examples 19 to 24, wherein determining whether the at least one criterion indicative of decreased impedance is satisfied includes comparing the impedance data generated during the first time interval to baseline impedance data generated by the implantable medical device prior to the first time interval.

Example 26: The method of example 25, wherein comparing the impedance data generated during the first time interval to baseline impedance data generated by the implantable medical device prior to the first time interval includes: determining a short-term moving average of impedance data generated by the implantable medical device including the impedance data generated during the first time interval; determining a long-term moving average of impedance data generated by the implantable medical device including the baseline impedance data; and accumulating differences between the short-term moving average and the long-term moving average.

Example 27: The method of any of examples 22 to 24 in combination with example 25 or 26 wherein determining whether the first one or more infection criteria are satisfied includes: determining a first indicator value based on one or more of the maximum temperature, the minimum impedance, or the difference between the time of the maximum temperature and the time of the minimum impedance; determining a second indicator value based on the comparison of the impedance data generated during the first time interval to the baseline impedance data; and determining an infection likelihood value based on application of a first weightage to the first indicator value and a second weightage to the second indicator value, wherein the first weightage is greater than the second weightage.

Example 28: The method of any of examples 19 to 27, wherein determining whether the second one or more infection criteria are satisfied includes determining whether a peak occurs in the temperature data generated during the second time interval.

Example 29: The method of any of examples 19 to 28, wherein determining whether the second one or more infection criteria are satisfied includes comparing the temperature data generated during the second time interval to baseline temperature data generated by the implantable medical device before the first time interval.

Example 30: The method of example 29, wherein comparing the temperature data generated during the second time interval to the baseline temperature data includes: determining a short-term moving average of temperature data generated by the implantable medical device including temperature data generated during the second time interval; determining a long-term moving average of temperature data generated by the implantable medical device including the baseline temperature data; and accumulating differences between the short-term moving average and the long-term moving average.

Example 31: The method of any of examples 19 to 30, wherein determining whether the at least one criterion indicative of increased impedance is satisfied includes comparing the impedance data generated during the second time interval to baseline impedance data generated by the implantable medical device before the first time interval.

Example 32: The method of any of examples 19 to 31, wherein comparing the impedance data generated during the second time interval to the baseline impedance data includes: determining a short-term moving average of impedance data generated by the implantable medical device including the impedance data generated during the second time interval; determining a long-term moving average of impedance data generated by the implantable medical device including the baseline impedance data; and accumulating differences between the short-term moving average and the long-term moving average.

Example 33: The method of any of examples 28 to 32, wherein determining whether the second one or more infection criteria are satisfied includes: determining a first indicator value based on one or more of the comparison of the temperature data generated during the second time interval to the baseline temperature data or the comparison of the impedance data generated during the second time interval to the baseline impedance data; determining a second indicator value based on the peak in the temperature data generated during the second time interval; and determining an infection likelihood value based on application of a first weightage to the first indicator value and a second weightage to the second indicator value, wherein the first weightage is greater than the second weightage.

Example 34: The method of any of examples 19 to 33, further includes determining at least one of an activity level or posture of the patient based on a signal from a motion sensor; and discarding at least a portion of at least one of the temperature data or the impedance data based on the activity level or posture.

Example 35: The method of any of examples 19 to 34, wherein the indication of infection is an indication of pocket infection.

Example 37: A system includes means for performing any of the methods of examples 19 to 36.

Example 38: A computer-readable storage medium including instructions that, when executed by processing circuitry, cause the processing circuitry to perform any of the methods of examples 19 to 36.

Various examples have been described. However, one skill in the art will appreciate that various modifications may be made to the described examples without departing from the scope of the claims.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, ROM, NVRAM, DRAM, SRAM, Flash memory, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Furthermore, although described primarily with reference to examples that provide an infection status to indicate a device pocket infection in response to detecting temperature changes in the device pocket, other examples may additionally or alternatively automatically modify a therapy in response to detecting the infection status in the patient. The therapy may be, as examples, a substance delivered by an implantable pump, a delivery of antibiotics, etc. These and other examples are within the scope of the following claims.

What is claimed is:

1. A system comprising:
an implantable medical device configured to generate temperature data and impedance data associated with temperature and impedance of a patient proximate to the implantable medical device; and
processing circuitry configured to:
determine whether a first one or more infection criteria are satisfied by temperature data and impedance data generated by the implantable medical device during a first time interval, wherein the first one or more infection criteria include at least one criterion indicative of decreased impedance;
determine whether a second one or more infection criteria are satisfied by the temperature data and impedance data generated by the implantable medical device during a second time interval subsequent to the first time interval, wherein the second one or more infection criteria include at least one criterion indicative of increased impedance; and
output, based on satisfaction of the first and second infection criteria, an indication of infection.

2. The system of claim 1, wherein the processing circuitry is configured to determine whether the one or more second infection criteria are satisfied in response to satisfaction of the one or more first infection criteria.

3. The system of claim 1, wherein the second time interval extends at least three days later than the first time interval.

4. The system of claim 1, wherein, to determine whether the first one or more infection criteria are satisfied, the processing circuitry is configured to determine a maximum temperature during the first time interval.

5. The system of claim 1, wherein, to determine whether the at least one criterion indicative of decreased impedance is satisfied, the processing circuitry is configured to determine a minimum impedance during the first time interval.

6. The system of claim 1, wherein, to determine whether the first one or more infection criteria are satisfied, the processing circuitry is configured to:
determine a maximum temperature during the first time interval;
determine a minimum impedance during the first time interval; and
determine a difference between a time of the maximum temperature and a time of the minimum impedance.

7. The system of claim 6, wherein, to determine whether the first one or more infection criteria are satisfied, the processing circuitry is configured to:
determine a first indicator value based on one or more of the maximum temperature, the minimum impedance, or the difference between the time of the maximum temperature and the time of the minimum impedance;
determine a second indicator value based on the comparison of the impedance data generated during the first time interval to the baseline impedance data; and
determine an infection likelihood value based on application of a first weightage to the first indicator value and a second weightage to the second indicator value, wherein the first weightage is greater than the second weightage.

8. The system of claim 1, wherein, to determine whether the at least one criterion indicative of decreased impedance is satisfied, the processing circuitry is configured to compare the impedance data generated during the first time interval to baseline impedance data generated by the implantable medical device prior to the first time interval.

9. The system of claim 8, wherein, to compare the impedance data generated during the first time interval to baseline impedance data generated by the implantable medical device prior to the first time interval, the processing circuitry is configured to:
determine a short-term moving average of impedance data generated by the implantable medical device comprising the impedance data generated during the first time interval;
determine a long-term moving average of impedance data generated by the implantable medical device comprising the baseline impedance data; and
accumulate differences between the short-term moving average and the long-term moving average.

10. The system of claim 1, wherein, to determine whether the second one or more infection criteria are satisfied, the processing circuitry is configured to determine whether a peak occurs in the temperature data generated during the second time interval.

11. The system of claim 10, wherein, to determine whether the second one or more infection criteria are satisfied, the processing circuitry is configured to:
determine a first indicator value based on one or more of the comparison of the temperature data generated during the second time interval to the baseline temperature data or the comparison of the impedance data generated during the second time interval to the baseline impedance data;
determine a second indicator value based on the peak in the temperature data generated during the second time interval; and
determine an infection likelihood value based on application of a first weightage to the first indicator value and a second weightage to the second indicator value, wherein the first weightage is greater than the second weightage.

12. The system of claim 1, wherein, to determine whether the second one or more infection criteria are satisfied, the processing circuitry is configured to compare the temperature data generated during the second time interval to baseline temperature data generated by the implantable medical device before the first time interval.

13. The system of claim 12, wherein, to compare the temperature data generated during the second time interval to the baseline temperature data, the processing circuitry is configured to:
determine a short-term moving average of temperature data generated by the implantable medical device comprising temperature data generated during the second time interval;
determine a long-term moving average of temperature data generated by the implantable medical device comprising the baseline temperature data; and
accumulate differences between the short-term moving average and the long-term moving average.

14. The system of claim 1, wherein, to determine whether the at least one criterion indicative of increased impedance is satisfied, the processing circuitry is configured to compare the impedance data generated during the second time interval to baseline impedance data generated by the implantable medical device before the first time interval.

15. The system of claim 1, wherein, to compare the impedance data generated during the second time interval to the baseline impedance data, the processing circuitry is configured to:
determine a short-term moving average of impedance data generated by the implantable medical device comprising the impedance data generated during the second time interval;
determine a long-term moving average of impedance data generated by the implantable medical device comprising the baseline impedance data; and
accumulate differences between the short-term moving average and the long-term moving average.

16. The system of claim 1, further comprising a motion sensor, wherein the processing circuitry is configured to:
determine at least one of an activity level or posture of the patient based on a signal from the motion sensor; and
discard at least a portion of at least one of the temperature data or the impedance data based on the activity level or posture.

17. The system of claim 1, wherein the indication of infection is an indication of pocket infection.

18. The system of claim 1, wherein the processing circuitry comprises processing circuitry of the implantable medical device.

19. A method comprising:
determining whether a first one or more infection criteria are satisfied by temperature data and impedance data generated by an implantable medical device during a first time interval, wherein the temperature data and impedance data are associated with temperature and impedance of a patient proximate to the implantable medical device, and wherein the first one or more infection criteria include at least one criterion indicative of decreased impedance;
determining whether a second one or more infection criteria are satisfied by the temperature data and impedance data generated by the implantable medical device during a second time interval subsequent to the first time interval, wherein the second one or more infection criteria include at least one criterion indicative of increased impedance; and
outputting, based on satisfaction of the first and second infection criteria, an indication of infection.

20. A computer-readable storage medium comprising instructions that, when executed by processing circuitry, cause the processing circuitry to:
determine whether a first one or more infection criteria are satisfied by temperature data and impedance data generated by an implantable medical device during a first time interval, wherein the temperature data and impedance data associated with temperature and impedance of a patient proximate to the implantable medical device, and wherein the first one or more infection criteria include at least one criterion indicative of decreased impedance;
determine whether a second one or more infection criteria are satisfied by the temperature data and impedance data generated by the implantable medical device during a second time interval subsequent to the first time interval, wherein the second one or more infection criteria include at least one criterion indicative of increased impedance; and
output, based on satisfaction of the first and second infection criteria, an indication of infection.
